(19) United States
(12) Patent Application Publication
(10) Pub. No.: US 2025/0282807 A1
(43) Pub. Date: Sep. 11, 2025

(54) PROCESS FOR ISOLATION AND/OR PURIFYING OF GLYCOLIPIDS

(71) Applicant: BIO BASE EUROPE PILOT PLANT VZW, Gent (BE)

(72) Inventors: Karolien MAES, Assenede (BE); Sophie ROELANTS, Melle (BE); Wim SOETAERT, Lievegem (BE); Emile REDANT, Aalter (BE); Bernd EVERAERT, Gentbrugge (BE)

(21) Appl. No.: 18/552,742

(22) PCT Filed: Mar. 30, 2021
(86) PCT No.: PCT/EP2021/058367

§ 371 Date: Sep. 27, 2023

(51) Int. Cl. C07H 1/08; C07H 3/04; C07H 15/10

(52) U.S. Cl. CPC C07H 1/08; C07H 3/04; C07H 15/10

(57) ABSTRACT

The present invention is directed to a process for isolating and/or purifying glycolipids from a glycolipid-containing composition wherein said process comprises the steps of: providing a process-unit with an adsorbent, wherein said adsorbent is a polymeric resin; contacting the glycolipid-containing composition with the adsorbent to load the polymeric resin with loaded material and wherein said loaded material comprises at least an amount of glycolipids; treating the adsorbent to recover a predetermined desired type of glycolipid present within the loaded material loaded on the adsorbent, wherein said treating comprises: contacting the adsorbent with a preselected recovering liquid to recover the predetermined desired type of glycolipid from the loaded material on the adsorbent; acquiring the recovered predetermined desired type of glycolipid from the process-unit. Said process can be used as an efficient purification method for biosurfactants, for example in the food or beverage industry, or in the cosmetic or pharmaceutical industry.

### CROSS-REFERENCES TO RELATED APPLICATIONS

This application is a national-stage application under 35 U.S.C. § 371 of International Application No. PCT/EP2021/058367, filed Mar. 30, 2021.

### TECHNICAL FIELD

The present invention is directed to a process to isolate and/or purify glycolipids from a glycolipid-containing composition. Said process can be used as an efficient purification method for biosurfactants, for example in the food, feed or beverage industry, or in the cosmetic, detergent, industrial and/or pharmaceutical industry.

### BACKGROUND

Biosurfactants such as glycolipids have a large market potential, however their success for bulk applications has been limited by economic limitations and purity requirements. For downstream processing, high yields, scalability, economic feasibility are imperative while purity is an important aspect certainly for specific applications. Hence, a large part of the manufacturing costs of biotechnological products is generally attributed to downstream processing costs and product losses.

The major challenges in downstream processing of biosurfactants is the complexity of the biosurfactant containing mixtures. Depending on whether the biosurfactants are produced by fermentation, biocatalysis, plant biomass or chemical synthesis, different kinds of impurities will be present in the complex mixture. These mixtures can comprise microbial cells, proteins, enzymes, salts, ions, peptides, carbohydrates, fatty alcohols, diols, fatty acids, oils and fats, non-desired biosurfactant byproducts or -precursors, etc. Proteins, peptides, enzymes could cause allergic reactions when they remain in the final product while genetically modified microorganism should not be detectable in the final product, nor should toxic catalysts. Also, lipophilic compounds should not remain in the final product, but it is notoriously difficult to separate amphiphilic compounds such as biosurfactants from lipophilic compounds typically present in the final mixtures as they are often used as substrates towards the production of biosurfactants and are often added in excess. Different types of impurities e.g. hydrophilic versus hydrophobic contaminants, often require different process units in the purification process which cause the total purification of the biosurfactants to be slow, expensive and may cause product loss in between the process units.

The required purity depends on the application of the target product. Pharmaceutical, food and cosmetic applications require an exceptional purity while fields of application such as (industrial) detergents, environmental remediation and agriculture may have less stringent requirements. Mostly it is mandatory to remove any cells, cell debris such as DNA and RNA, and proteins/peptides that might lead to possible allergic reactions. Depending on the application, it may even be essential to separate mixtures of glycolipids, purifying a specific glycolipid congener. Existing and applied purification methods for biosurfactants such as glycolipids are often a combination of techniques and processes, such as precipitation, melting, washing, solvent extraction, membrane filtration, foam separation, crystallization, ion exchange. However, such purification methods require different processing units and process steps resulting in product loss in each step.

### SUMMARY

The object of the present invention is to provide a technique to isolate glycolipids from various types of mixtures in a time- and space-efficient manner while achieving a preselected final product purity and high recovery/yield and with efficient use of resources.

The present invention therefore provides a process according to claim 1. More in particular, a process for isolating and/or purifying glycolipids from a glycolipid-containing composition, wherein said process comprises the steps of: —providing a process-unit with an adsorbent (R), wherein said adsorbent is a polymeric resin; —contacting the glycolipid-containing composition with the adsorbent to load the polymeric resin with loaded material (LM) and wherein said loaded material comprises at least an amount of glycolipids (LM1); treating the adsorbent to recover a predetermined desired type of glycolipid (PLM1), wherein said treating comprises: contacting the adsorbent with a preselected recovering liquid (RL) to recover the predetermined desired type of glycolipid from the loaded material on the adsorbent; acquiring the recovered predetermined desired type of glycolipid (PLM1) from the process-unit.

The invention is based on the insight that by using a well-designed process unit, one can select a predetermined type of glycolipid and isolate and/or purify said type of glycolipid from a complex mixture that is added to the process unit. It has been found that the polymeric resin can adsorb certain kinds of components and at least an amount of glycolipids from the mixture provided to the process unit. Due to the adsorbing capabilities of the polymeric resin, said components are loaded on the adsorbent and form the loaded material on the polymeric resin. Afterwards, by treating the adsorbent on which the loaded material is adsorbed with a preselected recovering liquid one can recover a predetermined desired type of glycolipid from the material loaded on the adsorbent. In this way, one can acquire the desired type of glycolipid from the mixture that is provided to the process unit in a way that a high purity and recovery can be achieved with very little or no additional downstream processing steps. In this way there is substantially less product loss and a high purity can be obtained, which is suitable for most applications without the need of cumbersome, slow and resource intensive additional treatment purification steps resulting in low final recoveries/yields and thus in suboptimal economics.

It is preferred that the process further comprises the step of: —washing the adsorbent with a washing liquid (WL) to separate at least non-loaded material (NLM) from the adsorbent; It has been found that by using washing liquids, the non-loaded material can be removed from the adsorbent.

A washing liquid can be understood as a liquid that is used to remove non loaded material NLM from the process unit. Namely the non-loaded material that is not adsorbed to the adsorbent, an exemplary washing liquid is RO water, but could equally consist of a solution comprising one or more washing liquids capable to remove non-loaded material from the process unit.

A recovery liquid can be understood as a liquid that is used to desorb and/or remove material from the adsorbent. The recovery liquid is typically used to desorb and recover a desired glycolipid. A recovery liquid can consist of a single liquid component or of a solution comprising one or more recovering liquids capable to desorb and/or remove material from the adsorbent

A treating liquid can be understood as a liquid that is used to treat the adsorbent so that certain fractions of secondary components such as proteins, pigments, fatty acids, salts, sugars, cells that are adsorbed to the adsorbent can be removed from the adsorbent and/or as a treating liquid that is used to modify loaded material, for example by partially hydrolyzing loaded material or by for example by performing (a) specific chemical modification(s) such as glycosylation on the loaded material. A treating liquid can consist of a single liquid component or of a solution comprising one or more treating liquids capable to modify loaded material and/or remove material from the adsorbent.

The loading capacity described herein can be understood as the amount of a target component, such as the desired type of glycolipid that binds to the adsorbent divided by the total amount of adsorbent. Said amounts are typically expressed by mass %, e.g kg of desired glycolipid adsorbed to the polymeric resin divided by the mass of the polymeric resin in kg.

Product purity can be understood as the amount of target component, such as the desired type of glycolipid, divided by the total amount of components present in the final product, and is typically expressed as mass %.

Throughout the present application reference is made to loaded and non-loaded materials. Within the context of the present invention loaded materials are materials that adsorb on the adsorbent. Non-loaded materials to the contrary do not adsorb on the absorbent.

Preferably, the treating-step further comprises the step of: —contacting the adsorbent with one or more treating liquids (TL) to modify at least an amount of glycolipids loaded on the adsorbent and/or remove one or more secondary components (NPLM2) from the adsorbent. In this way, one can remove said secondary components and purify the desired type of glycolipid and/or by modifying at least an amount of glycolipids loaded on the adsorbent, one can convert loaded material into the desired type of glycolipid. In some cases, the washing liquid and treating liquid are mixed so that the secondary components and the non loaded material can be removed at once e.g RO water mixed with isopropanol. Preferably, the one or more treating liquids (TL) are selected from: polar solvent, apolar solvent, alkaline solvent, acidic solvent, neutral solvent, or combination thereof. More specifically, it is preferred that the one or more treating liquids (TL) are selected from: methanol, ethanol, propanol, isopropanol, butanol, hexane, heptane, ethyl acetate, KOH, NaOH, NH₄OH, water, RO water or combinations thereof. By selecting the treating liquid one can treat the loaded material on the adsorbent and handle said loaded material in a predefined manner as will be further described herein.

It is preferred that the preselected recovering liquid (RL) is selected from the group comprising:
ionic liquids, liquid carbon dioxide, supercritical solvents, ethyl acetate, methanol, isopropanol, acetone, ethanol, heptane, ter-butyl-methyl ether, diethylether, acetonitrile, phenoxyethanol, benzylalcohol, phenetyl alcohol, hydrocinnamylalcohol, tetrahydrofurfuryl alcohol, dimethylisosorbide, methyl salicylate eugenol, linalool, hexanol, glacial acetic, dimethylcarbonate, certain glycolethers such as dipropyleneglycol methyl ether and 1-propoxy 2-propanol and lactate esters including ethyl-, butyl-, amyl-, ethylhexyl-lactate or combinations thereof. Said liquids have been found or are expected to have a desired capability to remove a predetermined type of glycolipid from the adsorbent. Preferably, the glycolipids (GL) are selected from the group comprising glycosylated fatty acids, glycosylated fatty alcohols, glycosylated carotenoids, glycosylated hopanoids, glycosylated sterols, glycosylated paraconic acids, glycoglycerolipids, glycosphingolipids, lipopolysaccharides, phenolic glycolipids, glycopeptidolipids, nucleoside lipids.

In an embodiment, the glycolipids (GL) are selected from the group comprising sophorolipids, rhamnolipids, cellobioselipids, xylolipids, trehalose lipids, mannosylerythritol lipids, glucolipids, fatty alcohol glucosides, alkyl poly glucosides, alkyl sophorosides, (anionic) alkyl glucosides, (anionic) alkyl pentosides, sucrose esters, sorbitan esters, methyl glucoside esters, fatty acid methyl glucamides, oligosaccharide fatty alcohols.

In an embodiment, the glycolipids (GLs) are selected from the acetylated and/or non-acetylated forms of: acidic sophorolipids (ASL), lactonic sophorolipids (LSL).

In a preferred embodiment, the glycolipids (GL) are selected from the acetylated and/or non-acetylated forms of: bola sophorolipids (BSL), bola sophorosides (BSS), alkyl sophorosides (ASS), alcohol glucosides (AGS), bola sophorosides (BSS), sucrose Esters (SE), bola glucosides (BGS), alkyl glucosides (ALGS), glucolipids, or combinations thereof.

In an embodiment of the invention, the treating-step comprises: contacting the adsorbent (R) with a treating solution (TS2) with a first concentration c1 of a treating liquid; contacting the adsorbent (R) with a recovering solution (RS2) with a second concentration c2 of a recovery liquid; wherein the first concentration c1 is such that that essentially all of the glycolipids (LM1) within the loaded material are not removed from the adsorbent and wherein the second concentration c2 is such that essentially all of the glycolipids (LM1) within the loaded material from the adsorbent are removed. In this way, one can first treat the adsorbent to remove other components different from the desired glycolipid and then recover the desired glycolipid with the recovering liquid.

In some embodiments the treating liquid and the recovering liquid can be the same. In said instances, the invention, the treating-step comprises: contacting the adsorbent with a treating solution with a first concentration c1 of a treating liquid; contacting the adsorbent with a recovering solution with a second concentration c2 of a recovery liquid; wherein the first concentration is lower than the second concentration such that c1 of the treating liquid is low enough so that glycolipids within the loaded material is not efficiently removed from the adsorbent and c2 of the recovery liquid is high enough to efficiently remove glycolipids within the loaded material from the adsorbent. By choosing the concentration of the recovering liquid, one can select and remove certain secondary components from the adsorbent with the recovering solution using a lower concentration c1, wherein said lower concentration does not elute a substantial amount of the desired glycolipids from the adsorbent. In this way, one can remove impurities such as specific proteins and pigments so that that the desired glycolipid remains mainly on the adsorbent in a purified manner. Said purified desired glycolipid can then be acquired by the recovering liquid with a higher concentration c2.

In a preferred embodiment wherein the treating liquid and the recovering liquid are the same, the impurities are removed with a recovering liquid with a concentration of lower than 45%, more preferably a concentration between 10-35%, most preferably with a concentration in the range of 15-30%. Preferably, the recovering liquid comprises isopropanol diluted with RO water.

In an embodiment of the invention, the treating liquid and the recovering liquid are both one of:

a polar solvent, an apolar solvent, an alkaline solvent, an acidic solvent, a neutral solvent, or a combination thereof.

In an aspect of the invention, the process is used to remove a first fraction of loaded and/or non loaded one or more secondary components from the adsorbent wherein said first fraction is selected from: impurities such as proteins, pigments and the like or undesired types of glycolipids. In this way, said one or more secondary components which are typically impurities or other types of glycolipids, are removed from the desired glycolipid.

In an embodiment of the invention, the treating-step comprises: contacting the adsorbent with a first treating liquid, preferably an alkaline reagent, to at least partially hydrolyze/convert an amount of glycolipids loaded on the adsorbent. In this way, one can convert certain types of glycolipids such as glycolipids comprising e.g. ester functionalities into desired types of glycolipids by partial hydrolysis of the ester functionality. Due to the conversion, the desired type of glycolipid can thus either be created or can be purified. In an aspect of the invention, one can use the process according to the invention to convert acetylated glycolipids into non-acetylated glycolipids. In another aspect, one can use the process to convert acetylated and/or non-acetylated lactonic sophorolipids into acetylated and/or non-acetylated acidic sophorolipids. In another aspect, one can use the process to convert acetylated and/or non-acetylated bola sophorolipids into acetylated and/or non-acetylated acidic sophorolipids and acetylated and/or non-acetylated sophorose.

In an embodiment of the invention, the treating-step comprises contacting the adsorbent with a second treating liquid, preferably an apolar solvent, more preferably an apolar organic solvent, to remove a second fraction of the one or more secondary components from the loaded material; wherein said second fraction is selected from: apolar components such as free fatty acids, fatty alcohols, alkanes and/or oils, etc. Preferably, the treating liquid is chosen from hexane, heptane or methyl tert-butyl ether. Preferably, the treating liquid is chosen from hexane or heptane. The second treating liquid may further be mixed with a polar solvent such as methanol, butanol, water, isopropanol or combinations thereof. By using said second treating liquid, the second fraction can be removed so that the desired glycolipids loaded on the adsorbents are purified.

In an embodiment of the invention, the treating-step comprises contacting the adsorbent with a third treating liquid, preferably a polar solvent, to remove a third fraction of the one or more secondary components from the loaded material; wherein said third fraction is selected from: hydrophilic impurities such as sugars, proteins, carbohydrates, salts and the like. By using said third treating liquid, the third fraction can be removed so that the desired glycolipid loaded on the adsorbents can be purified.

Each of the foregoing treating steps can be performed in isolation or in combination with one or more of said treating steps. In a preferred embodiment all the treating steps are performed. By selecting said first, second and third treating liquids one can selectively remove certain fractions from the mixture that is provided the to process unit as well as from certain fractions from the loaded material on the adsorbent. In this way, one can use the process unit to isolate and/or purify the desired glycolipids to a high degree of purity, preferably in the range of more than 90%, even more preferably more than 93% even without having to rely on further purification steps.

In an embodiment of the invention the adsorbent is a neutral polymeric resin. In this way the desired glycolipid bind to the neutral polymeric resin due to a hydrophobic interaction. The overall charge of the neutral polymeric resin can be neutral and no ion exchange is needed to bind the desired glycolipid to the polymeric resin. The hydrophobic interaction or affinity of the desired glycolipid may be altered by altering the temperatures. Preferably the adsorbent is a polymeric resin and is selected from the group comprising polymethacrylate resin, acrylic resin, polystyrene resin, or a combinations thereof; in particular selected from: Styrene-divinylbenzene, Polymethacrylate, Chemically brominated polystyrene, Acrylic ester, polystyrene, crosslinked polystyrene, Methacrylic, Porous polystyrene-divinylbenzene, Styrene-divinylbenzene, Styrene-divinylbenzene, Acrylic ester, Polymethacrylate, Chemically brominated polystyrene. Said selected resins have been found to have a desired affinity with glycolipids so that at least an amount of glycolipids bind to the polymeric resin upon contact. Due to a high affinity, high loading capacities can be achieved.

In an embodiment of the invention the process unit has an input stream (S1) and an output stream (S2); It is preferred that the washing step is carried out by inputting the washing liquid (WL) via said input stream to the process-unit and until output stream parameters are within a predetermined range of input stream parameters. In an embodiment the treating step is carried out by inputting the treating liquid (TL) via said input stream to the process-unit and until output stream parameters are within a predetermined range of input stream parameters. For example, inputting RO water to remove hydrophilic impurities such as sugars, proteins, peptides, polyols, organic acids, inorganic acids, carbohydrates, salts. In an embodiment, said parameters are chosen from: conductivity, refractive index, pH, protein content, sugar content, test-swab values or a combination thereof. Naturally, when for example considering conductivity, the washing step or treating step is carried out until the conductivity of the output stream reaches a value which is lower than 150% of the conductivity of the input stream.

In an embodiment of the invention, the process further comprises the steps of: —evaporating the recovering liquid; —evaporating the treating liquid if present; By evaporating the recovering liquid and/or treating liquid which was used to recover a desired type glycolipid from the adsorbent, one can remove and preferably reuse said recovering and/or treating liquid. In this manner one can obtain the desired glycolipid in a highly pure form. It has been found that purities of more than 98% by mass can be reached. In an embodiment of the invention, the process comprises the step of recycling at least one of: recovering liquid (RL), treating liquid (TL), and optionally washing liquid (WL). Practical tests have shown one can further efficiently use resources by recycling the recovering liquids and/or treating liquids and optionally the washing liquids.

It is preferred that at least one of: recovering liquid (RL), treating liquid (TL), and optionally washing liquid (WL) are recycled. In this way, efficient use of resources can be improved.

In an embodiment, the glycolipid-containing composition is the end-product of a plant extract or fermentative, enzymatic, plant biomass or chemical production method. In an embodiment, the process is performed in combination with a fermentation process; preferably in combination with a fermentation process in an in-situ product recovery set up.

According to an embodiment the process is performed in a batch mode, in a continuous mode or in a semi-continuous mode. Preferably a continuous mode or semi-continuous mode.

According to an embodiment, the glycolipid-containing composition is passed through a reactor that comprises the adsorbent.

According to an embodiment the glycolipid-containing composition is passed over a column packed with the polymeric adsorbent.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 illustrates a flowchart comprising steps of the process.

FIG. 2 shows an exemplary embodiment of the process.

### DETAILED DESCRIPTION

The present application is directed to a process for the isolation of glycolipids from a glycolipid-containing composition. Typically, said composition is derived biological origin. Said process is based on the steps according to the claim 1 and will be further explained below.

The isolated glycolipids can be used as biosurfactants and show unique properties, such as mild production conditions, lower toxicity, higher biodegradability and skin- and environmental compatibility, (complete) biobased nature, etc. compared to the classic counterparts which are typically fossil derived. Microbial biosurfactants in particular have some advantages because they can be produced from local (waste) biomass streams thus avoiding the dependence on tropical (e.g. palm oil) and/or fossil resources associated with clear issues. These numerous advantages have prompted applications not only in the food, agrochemical, cosmetic and pharmaceutical industries, but in environmental protection and energy-saving technology as well. Although biosurfactants have a large market potential, their success for bulk applications has been limited by economic and technical limitations and stringent purity requirements for some markets. In upstream processing, the selective and efficient (high yield on substrate) conversion of cheap raw materials and high productivities are required. For downstream processing, high yields (recoveries), are imperative. In many applications, the purity of the desired glycolipid is of importance and secondary components such as allergens should be removed. In many cases, the majority of the manufacturing costs of biotechnological products can be attributed to downstream processing costs and corresponding product losses.

Some major challenges in the downstream processing of biosurfactants are associated with the complexity of the mixtures, which often contain relatively low desired product concentrations. Depending on whether the biosurfactants are produced through fermentation, biocatalysis, plant biomass or by chemical synthesis, different impurities will be present in the mixture. The various production methods generally require a hydrophilic substrate (e.g. glycerol, glycose, sucrose, etc.) as well as a hydrophobic substrate (e.g. alkane, fatty acid, triacyl glycerides, fatty alcohols, etc.) to produce the glycolipid structure. Although some biochemical production processes such as a fermentation production process do not strictly require that both hydrophilic and hydrophobic substrates are provided, the combination of the substrates generally results in a higher productivity such that this is the preferred option.

A fermentation production method typically results in a complex mixture since a fermentation broth can contain substrates, cells, salts, carbohydrates, polyols, proteins, peptides and intra- and extracellular metabolites and the like. The hydrophobic substrates, e.g. oils and fatty acids are often solubilized in the broth by the produced biosurfactants.

The major challenges in the processing of biosurfactants thus lies in the complexity of the mixtures, which often contain relatively low product concentrations, and the presence of a myriad of impurities in said mixtures. Impurities such as any cells, debris, DNA, RNA and proteins need to be removed. In particular due to possible allergic reactions caused by proteins, peptides, or enzymes and DNA/RNA potentially derived from GM organisms. To date, specific isolation techniques such as chromatographic and/or adsorption have often mostly been limited to the isolation and purification of low-concentrated biosurfactant containing mixtures in R&D laboratory set ups, because of quite inefficient processes the low adsorption capacity of resins, the size of the apparatus, and the large investment and operation costs.

Frequently it is stated in the art that low adsorption capacity of prior art adsorbents and the lack of separation capability between the biosurfactants and the other components in the mixture, high costs of the resins, and large size of required apparatuses make adsorption unsuitable for most applications. Additionally, the use of adsorption as a purification method for the purification of (fermentatively produced) glycolipids such as acetylated or non-acetylated forms of: bola sophorolipids (BSL), bola sophorosides (BSS), alkyl sophorosides (ASS), alcohol glucosides (AGS), sucrose Esters (SE), bola glucosides (BGS), alkyl glucosides (ALGS), glucolipids has not been reported.

With the present invention, the inventors describe an improved process for the isolation of glycolipids from a mixture comprising glycolipids resulting in high-purity products with high recoveries, and hereby opening up the use of the isolated glycolipids for several applications with a minimized risk of allergic reactions. In addition, the desired purity of the isolated glycolipids can be modified with flexible and washing and/or treating steps. Furthermore, the glycolipids isolated with the process of the present invention can further be modified during the purification process, which eliminates the need for additional processing and/or purification steps in (downstream) process units that might be required when derivatizing and subsequently purifying glycolipids in conventional methods. As such, the process of the present invention results in more efficient methods for conversion and/or isolation of biosurfactants.

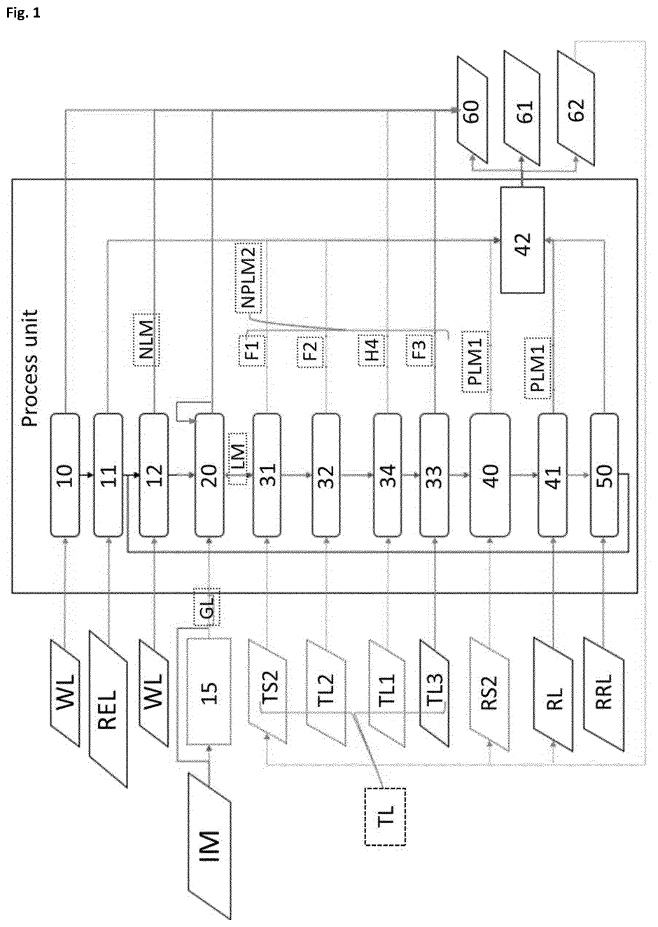

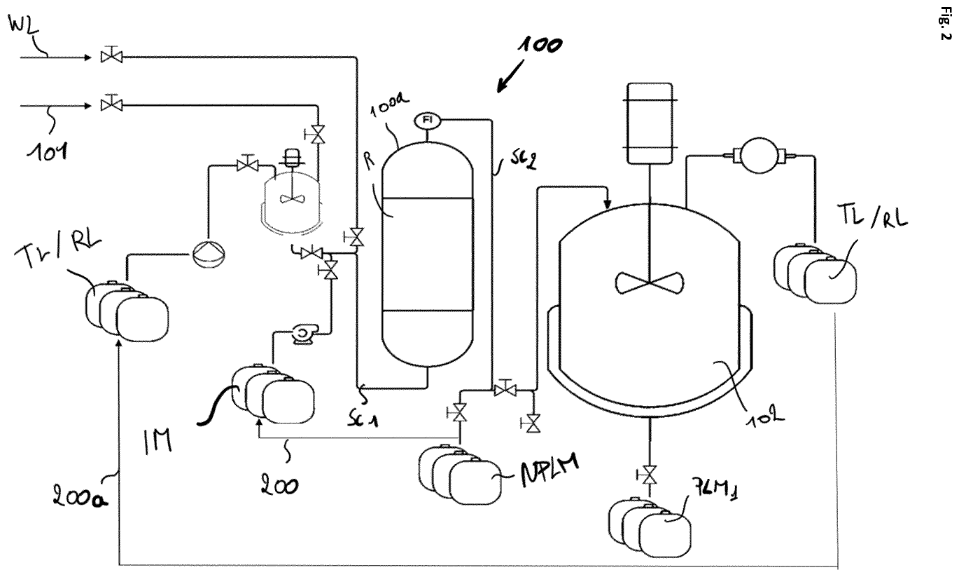

An exemplary embodiment of the process according to the invention will be explained in more detail with use of the step flowchart shown in FIG. 1 and the process flow chart shown in FIG. 2. The process unit comprises a polymeric resin as an adsorbent R.

As used herein, the terms complex mixture, glycolipid containing composition, input mixture can be used interchangeably, unless the context describes or implies otherwise.

As shown in the step flowchart of FIG. 1, the process may begin with an equilibration step 10. A washing liquid WL is typically used to equilibrate the polymeric resin. A preferred washing liquid is reverse osmose water, also known as RO water. The washing liquid WL is typically added in 3 to 5 bed volumes. In addition or as alternative, a resin equilibrium step 11 is carried out to wherein the polymeric resin is subjected to a resin equilibrium liquid REL. Typically the equilibrium liquid is similar to or the same as the recovering liquid which is used later in the process to recover the desired type of glycolipid. The washing liquid is used to remove non loaded material NLM from the process unit. Namely the non loaded material that is not adsorbed on to the adsorbent. A complex mixture IM comprises the glycolipids GL and is provided to the process unit. The mixture may be pre-treated with a pretreatment step 15, such as a microfiltration step, a gravimetrical separation step or a centrifugation step. The glycolipids are loaded on the adsorbent 20 and remaining non-loaded material can be removed via a waste stream 60, an optional recycle stream can be added in between for efficient use of resources.

After the loading step 20, the adsorbent can be subjected to a series of treatment steps (31 to 34) using treating liquids TL to remove secondary components NPLM2 from the adsorbent. Typical secondary components that are removed are selected from the group comprising ions, salts, saccharides, (oligo) saccharides, cells, oil, fatty acids, proteins, peptides, amino acids, residual hydrophobic substrates and/or hydrophobic substances, pigments or a combination thereof.

The treating liquids TL can be added back to the process unit via a recycle stream 62 for efficient resource use. The treating liquids TL are preferably selected from: isopropanol, methanol, ethanol, propanol, butanol, hexane, heptane, ethyl acetate, NaOH, NH₄OH, water, RO water or combinations thereof. By selecting a predetermined treating liquid TL, one can remove a predetermined fraction of loaded material NPLM2 from the adsorbent. In this way, the desired glycolipid can be purified. The desired glycolipid can than be recovered by the recovering liquid RL. When the recovering liquid RL is removed during purification step 42, one can obtain the desired glycolipid in a highly pure form 61.

A feature of the process of the present invention is that several treating steps can be present and that all treating steps can be performed on the same process unit. In said instance, the adsorbent on which the glycolipids are adsorbed can be treated with water, buffer solution, solvent or mixtures thereof, to remove specific impurities. An aqueous wash is typically sufficient to remove hydrophilic impurities present in the fraction that is not adsorbed onto the adsorbent. Such impurities are various organic or inorganic salts and -acids, sugars, carbohydrates, polyols, w proteins, peptides and various pigment/colour components. Washing with buffer solutions can improve the washing out of badly water soluble compounds or to help ensuring the stability of the product. Washing with water-solvent mixtures (such as, but not limited to methanol, ethanol, isopropanol and acetone) can aid with the washing out of impurities that are otherwise weakly adsorbed onto the adsorbent.

In treating step 31 the adsorbent is treated with a treating solution (TS2) to remove a first mfraction F1 form the adsorbent. It has been found that when the treating liquid that is normally capable of eluting the desired glycolipids from the adsorbent, has a lower concentration than the one used to elute the desired glycolipids from the adsorbent, one can remove impurities such as proteins and colour pigments from the adsorbent while the desired glycolipid mainly remains on the adsorbent. It this way, the desired glycolipid can be purified. Namely, since the first concentration is not high enough to efficiently desorb the desired glycolipid, said desired glucolipid remains on the adsorbent A preferred treating solution is an alcohol-solvent solution, wherein the alcohol is a C1-C7 alcohol diluted in an aqueous solution, such as water, preferably in a concentration of 20-40 vol. %, more preferably 24-34 vol. % most preferably 25-30 vol. %. A preferred treating solution is propanol, even more preferred isopropanol. The desired glycolipid can be removed afterwards in a recovery step 40 by recovering the desired type of glycolipid PLM1 with a recovering solution RS2. The recovering solution can be similar to the treating solution but with a higher concentration that is high enough to remove the desired glycolipid. Process wise, it is advantageous when similar or the same type of solution is used.

The inventors also have found that the adsorbent can be subjected to a treatment step 32 wherein the adsorbent is treated with a second treating liquid TL2, preferably an apolar organic solvent. In this way, one can use the apolar organic solvent to remove a second fraction F2 comprising apolar components from the adsorbent. In this way, components such as free fatty acids, fatty alcohols, diols, dicarboxylic acids, triacyl glycerides, oils etc. can be removed from the adsorbent while the desired glycolipid remains on the adsorbent. By removing the second fraction, the desired glycolipid can be acquired later in a purer form by subjecting the adsorbent to the recovering liquid to recover the desired glycolipids. Hexane and heptane are preferred organic solvents. Treating with pure or mixtures of non-polar solvents such as, but not limited to, hexane, heptane, cyclohexane, and diethylether), in which the glycolipids are badly soluble, can be used to wash out impurities that are adsorbed to the polymeric adsorbent. This is particularly useful for the separation of hydrophobic substrates such as free fatty acids, oils, fatty alcohols, fats, diols, dicarboxylic acids, triacyl glycerides, etc. of which some are used as substrates in the production of glycolipids and/or are co-produced by the microbial production strain, in case these impurities co-elute with the glycolipids.

A further treating step 33 can be used to purify the desired glycolipid. The inventors have found that further purification of the desired glycolipid can be obtained by treating the adsorbent during treating step 33 with a third treating liquid TL3, preferably a polar solvent, such as RO water. In this case, it has been found that also hydrophilic impurities such as such as sugars, proteins, peptides, organic acid, polyols, carbohydrates, salts and the like, can be removed from the adsorbent while the desired type of glycolipid remains loaded on the adsorbent.

It has further surprisingly been found that treating step 34 can be used for modifications and/or conversions of the glycolipid when said glycolipid is adsorbed on the adsorbent. Examples of such a modification include chemical hydrolysis of ester- and glycosidic bonds; or esterification, glycosylation and other chemical modifications such as (bio) chemical derivatisation routes described in the art are, but not limited to: glycosylation, acylation, alkylation, amidation, amination, arylation, biotinylation, 15 carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation, thionation, or any combination thereof towards for example, but without limitation, ω-quaternary ammonium SLs (QASLs), ω-SS amine oxides, ω-SS amines, ω20 bolamphiphile SSs, etc. such as for example described in the art for wild type SLs described by (Delbeke, 2016; Delbeke et al., 2018; Delbeke, Lozach, et al., 2016; Delbeke, Movsisyan, et al., 2015; Delbeke, Roelants, et al., 2016; Delbeke, Roman, et al., 2015; D. Develter & Fleurackers, 2008; Gross et al., 2013; Van Bogaert et al., 2011). Such modifications can be performed by passing an aqueous solution or solvent containing the (bio) catalyst optionally combined with donors/acceptors required in the (bio) chemical reaction over the adsorbent, during which the (bio) catalyst reacts with the adsorbed glycolipid.

In a preferred embodiment, the adsorbent and the loaded material thereon is contacted with an alkaline agent to partially modify an amount of glycolipids present in the loaded material. In this way, one can use the modifications and/or conversions to further purify desired type of glycolipid by converting other types of glycolipid on the adsorbent into the desired type of glycolipid. Due to the conversion, the desired type of glycolipid can either be created or can be purified. One can use the process to convert acetylated glycolipids into non-acetylated glycolipids and/or lactonic glycolipids into acidic glycolipids. Non-acetylated lactonic sophorolipids can be converted into acetylated and/or non-acetylated acidic sophorolipids. In another aspect, one can use the process to convert acetylated and/or non-acetylated bola sophorolipids into acetylated and/or non-acetylated acidic sophorolipids and acetylated and non-acetylated sophorose.

FIG. 2 illustrates an exemplary embodiment of a process flowchart. The process unit 100 is provided with an adsorbent R. In the exemplary embodiment shown, the adsorbent is provided in a column 100a with input stream SC1 and output stream SC2. The washing liquid WL, treating liquids TL and recovering liquids RL can be provided to the reactor in a controlled flow which can be continuous or semi continuous. The lines can be pressurized with a pressurization gas such as N2-gas. In addition, or as an alternative, pumps such as membrane pumps can be used to control the liquids provided to the adsorbent. In an embodiment, a plurality of columns 100a can be used, the columns can be placed sequentially or in parallel with each other. During start up, the adsorbent R is typically screened with the washing liquid WL before the glycolipid containing mixture IM is provided to the column 100a. The glycolipid containing mixture is supplied to the column 100a to load the adsorbent with glycolipids. By selecting a type of recovering liquid RL one can recover and isolate a desired type of glycolipid by desorbing the glycolipid with the recovering liquid RL. The recovering liquids and/or treating liquids can be recycled by a recycle stream 200a.

The present invention is typically characterized in that it provides a process to isolate glycolipids from a glycolipid-containing composition IM. Said composition can be provided as an input mixture IM to the process unit of the process. In an embodiment, said glycolipid-containing composition is the end-product of a fermentation process, an enzymatic, plant biomass or a chemical production method.

In a further embodiment, said glycolipid-containing composition is the end-product of plant extraction. In another embodiment, the glycolipid-containing composition in the process according to the different embodiments of the invention is the end-product of an enzymatic derivatization process. In another embodiment, the glycolipid-containing composition in the process according to the different embodiments of the invention is the end-product of chemical derivatization process. Derivatized through chemical (derivatization) routes described in the art are, but not limited to: fisher synthesis, glycosylation, acylation, alkylation, amidation, amination, arylation, biotinylation, 15 carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation, thionation, or any combination thereof towards for example, but without limitation, w-quaternary ammonium SLs (QASLs), ω-SS amine oxides, ω-SS amines, ω20 bolamphiphile SSs, etc. such as for example described in the art for wild type SLs described by (Delbeke, 2016; Delbeke et al., 2018; Delbeke, Lozach, et al., 2016; Delbeke, Movsisyan, et al., 2015; Delbeke, Roelants, et al., 2016; Delbeke, Roman, et al., 2015; D. Develter & Fleurackers, 2008; Gross et al., 2013; Van Bogaert et al., 2011). T.

In a particular embodiment, the process according to the present invention is to isolate polymeric glycolipids from a glycolipid-containing composition IM. In yet another embodiment, the glycolipids are selected from the group comprising glycosylated fatty acids, glycosylated fatty alcohols, glycosylated carotenoids, glycosylated hopanoids, glycosylated sterols, glycosylated paraconic acids, glycoglycerolipids, glycosphingolipids, lipopolysaccharides, phenolic glycolipids, glycopeptidolipids, nucleoside lipids. In an even further embodiment, the process according to the present invention is to isolate sophorolipids, rhamnolipids, xylolipids, trehalose lipids, mannoseylerythritol lipids, glucolipids, fatty alcohol glucosides, alkyl poly glucosides, alkyl sophorosides, (anionic) alkyl glucosides, (anionic) alkyl pentosides, sugar esters, fatty acids methyl glucamides, oligosaccharide fatty alcohols from a glycolipid-containing composition, wherein said specific glycolipids are adsorbed on a polymeric adsorbent, and wherein said specific glycolipids are subsequently desorbed from said polymeric adsorbent. In still a further specific embodiment, the present invention discloses a process to isolate sophorolipids from a sophorolipid-containing composition, said process comprising adsorption of the sophorolipids on a polymeric adsorbent, and desorption of the sophorolipids from said polymeric adsorbent.

In a preferred embodiment, the glycolipid composition comprises glycolipids such as for example bola glucosides, acetylated bola glucosides, alkyl sophorosides, acetylated alkyl sophorosides, alkyl glucosides, acetylated alkyl glucosides, alcohol glucosides, acetylated alcohol glucosides, bola sophorosides, acetylated bola sophorosides, as described in WO2020104582A1 and incorporated herein by reference, and bola sophorolipids as described in WO2015028278A1 and incorporated herein by reference

In an embodiment, a chemically brominated polystyrene-based resin is used to adsorb glycolipids from the input mixture IM. An alcohol solvent is used as a recovering liquid to selectively desorb bola SL. In this way, one can isolate bola SL from other kinds of glycolipids such as acidic bola SL.

In an embodiment, acidic SLs are isolated by removing other components from the adsorbent such as secondary components and/or other types of glycolipids such as bola SLs. The other types of glycolipids are removed, and preferably recovered by a recovering liquid RL, such as isopropanol, preferably a non-acidic recovering liquid. Then the acidic SL is recovered by an acidic recovering liquid, wherein the acidic recovering liquid is an aqueous solution comprising an acetic acid, a citric acid and or a mixture comprising a hydrogen halide such as hydrochloric acid, or mixtures thereof.

In an embodiment, apolar, low-water soluble SL, such as lactonic SL is isolated from a complex mixture by absorbing the apolar SL on an adsorbent, preferably a polymeric resin, more preferably a polymethacrylate resin and/or acrylic ester-based resin.

In an embodiment, the input mixture IM comprises fermentation broth dissolved in an alcohol solution, such as an ethanol solution. Preferably the alcohol solution comprises a C1, C2, C3, C4 alcohol with a concentration of 80% and higher.

In an embodiment of the invention, alkyl sophorosides (ASS) are recovered from the absorbent R with a recovering liquid comprising a solvent mixture of a low polar solvent and an apolar solvent. The low polar solvent preferably has a with a water solubility between 5 and 80 g/100 mL, preferably between 7 and 80 g/100 mL, most preferably between 7 and 80 g/100 mL measured at 20° C. The apolar solvent preferably has a water solubility between 0 and 5 g/100 mL, preferably between 0 and 1 g/100 mL measured at 20° C. The skilled person can consult the water solubility parameters according to the ILO international chemical safety cards. A preferred apolar solvent is an alkane such as hexane, octane and heptane. A preferred low polar solvent is an ester such as ethyl acetate. The mixture preferably further comprises an alcohol, preferably any one of a C1, C2, C3, C4-alcohol, e.g methanol and/or butanol.

The glycolipid-containing composition IM can further comprise any additional compounds or substances. In a further embodiment, the glycolipid-containing composition comprises glycolipids and one or more additional secondary compounds selected from the group comprising ions, salts, (oligo) saccharides, cells, organic and/or inorganic acids, oil, fatty acids, proteins, enzymes, peptides, amino acids, (residual) hydrophobic substrates, residual hydrophobic substances, pigments and/or allergens or a combination thereof.

The process according to embodiments of the invention is characterized in that secondary components such as proteins, peptides, (residual) hydrophobic substrates, residual hydrophobic substances, salts, and/or organic acids are removed from the glycolipid-containing composition before desorption of the glycolipids from the polymeric adsorbent. Said (residual) hydrophobic substances can be selected from fatty acids, fatty alcohols, fatty diols, dicarboxylic acids, triacyl glycerides, alkanes, oils, fats, etc. It has been found that said components can be removed by treating liquid TL.

Typical for the present invention is that glycolipids are first adsorbed to the polymeric adsorbent R, followed by a desorption of the glycolipids from said polymeric adsorbent. In particular, desorption of the glycolipids is performed with a recovering liquid RL which can be a recovering solution comprising one or more solvents; preferably one or more organic or anorganic solvents. The inventors found that adsorption of the glycolipids on the polymeric adsorbent is based on the affinity between the glycolipid and the adsorbent. The adsorbent is a polymeric resin. It has been found that certain polymeric resins can achieve higher loading capacities, in particular loading capacities of more than 5% and even up to 10% or more. Preferred polymeric resins are selected from the polymeric resin is selected from the group comprising polymethacrylate resin, acrylic resin, polystyrene resin, or a combinations thereof; in particular selected from: Styrene-divinylbenzene, Polymethacrylate, Chemically brominated polystyrene, Acrylic ester, polystyrene, crosslinked polystyrene, Methacrylic, Porous polystyrene-divinylbenzene, Styrene-divinylbenzene, Styrene-divinylbenzene, Acrylic ester, Polymethacrylate, Chemically brominated polystyrene.

The process according to its different embodiments, is further characterized in that the desorption of the glycolipids is preferably performed with a recovering liquid RL comprising one or more solvents; preferably one or more organic or anorganic solvents. In a further embodiment, said one or more solvents are selected from the group comprising ionic liquids, liquid carbon dioxide, supercritical solvents, ethyl acetate, methanol, isopropanol, acetone, ethanol, heptane, ter-butyl-methyl ether, diethylether, acetonitrile, phenoxyethanol, benzylalcohol, phenetyl alcohol, hydrocinnamylalcohol, tetrahydrofurfuryl alcohol, dimethylisosorbide, methyl salicylate eugenol, linalool, hexanol, glacial acetic, dimethylcarbonate, certain glycolethers such as dipropyleneglycol methyl ether and 1-propoxy 2-propanol and lactate esters including ethyl-, butyl-, amyl-, ethylhexyl-lactate. In another embodiment of the present invention, the total concentration of said one or more solvents as mentioned above is between 20% and 100%; preferably between 50% and 100%; even more preferably between 70% and 100%, wherein said concentrations are expressed as volumetric concentrations. Further, the recovering of the glycolipid from the polymeric adsorbent can be performed by desorption or elution by passing a recovering liquid, in particular a water-solvent mixture or pure solvent over the adsorbent. Here it is imperative to select a solvent in which the glycolipids have a sufficiently high solubility. The efficiency of the desorption and hence elution depends on the contact time, the temperature, the solubility of the glycolipid in the solvent/eluent, the volume of the solvent/eluent, and the specific matrix of the polymeric adsorbent.

The process according to the different embodiments of the invention is typically characterized in that during isolation of the glycolipids from the composition, the glycolipids are adsorbed on a polymeric adsorbent. In a particular embodiment, said polymeric adsorbent is a polymeric adsorption resin. In yet another embodiment, said polymeric adsorbent is selected from the group comprising polymethacrylate resin acrylic resin, polystyrene resin, or a mixture thereof. In still a further specific embodiment, the polymeric adsorbent is polystyrene resin.

In another aspect of the invention, the process further comprises one or more treating steps. Preferably, at least one treating step is performed after the adsorption of the glycolipids on a polymeric adsorbent and before the desorption of the glycolipids from said polymeric adsorbent. The wash steps in the process of the invention can be performed using water, a buffer solution, a solvent, or a combination of those.

In addition, using different treating solutions with varying hydrophobicity, a gradual desorption of the loaded material can be obtained. For example acidic and/or lactonic sophorolipids and/or a mixture thereof can be separated from fatty acids or different congeners of sophorolipids can be separated from each other e.g. separation of bola SLs from acidic SLs.

The process according to the present is to isolate glycolipids from a glycolipid-containing composition, wherein adsorption of the glycolipids on a polymeric adsorbent is followed by desorption of the glycolipids from said polymeric adsorbent. In a particular embodiment, and during the process, a (bio) chemical and/or enzymatic modification of the adsorbed glycolipids can occur before desorption of the glycolipids from the polymeric adsorbent by using treating liquids TL. The chemical and/or enzymatic modification of the adsorbed glycolipids can be selected from chemical and/or enzymatic hydrolysis of ester bonds, chemical and/or enzymatic hydrolysis of glycosidic bonds, esterification, etherification, glycosylation, polymerisation, amidation, (reductive) amination, quaternization, oxidation, epoxidation. In this way, one can alter the glycolipids that are loaded on the absorbent and convert at least an amount of the loaded glycolipids into a desired type of glycolipid in the same process unit without having to rely on additional modification and/or downstream steps. In this way, one can both modify and purify a desired glycolipid in the same process unit. In addition, by modifying the loaded material into the desired glycolipids, the purity of said desired glycolipid can be improved.

At the end of the isolation process, the desired glycolipid is for example collected from the eluted fraction by evaporation, preferably vacuum evaporation, or by ultra- or nanofiltration in combination with diafiltrations. As illustrated in FIG. 2, the treating liquids and/or recovering liquids can be separated from the desired glycolipids PLM1 in the evaporation container 102. The pressure in the container 102 filled with liquid is reduced below the vapor pressure of the treating and/or recovering liquids, causing said liquids to evaporate at a lower temperature than normal. The evaporated liquids are preferably recycled back to the beginning of the process for optimal resource use. Tests have shown that the treating and/or recovery liquids can be reused in this way one achieves an efficient use of resources.

In another aspect, the polymeric adsorbent R can be regenerated and prepared for a next round of isolation. The resin can be regenerated in a quasi-infinitive fashion. Tests have shown that the resin can be regenerated such that the resin can be reused for several process runs which is beneficial in view of efficient resource use. The regeneration can be performed with a combination of water, buffer and/or solvent. Although this washing step is not strictly required, it may be advantageous in case impurities accumulate on the adsorbent which may reduce its loading capacity.

In still another aspect of the present invention, the process to isolate glycolipids from a glycolipid-containing composition is performed in a batch mode. In another embodiment, the process is performed in a continuous mode. In still another embodiment, the process is performed in a semi-continuous mode.

In a further aspect, the process according to the invention is performed in combination with a fermentation, biocatalytic and/or chemical process. In said aspect, production of the glycolipid-containing composition by fermentation is performed in combination with the isolation of the glycolipids, for example in combination with fermentation in an in situ product recovery set up. In a specific setup, the polymeric adsorbent and the glycolipid-containing composition are mixed in a reactor. In a further embodiment, and in said setup, the process according to the invention takes place in a reactor.

In an embodiment, the glycolipids loaded on the absorbent are chemically modified by partial hydrolysis. In a specific embodiment, bola sophorolipids (Bola SL) where used to produce acidic sophorolipids SL and sophorose as for example described in WO2015028278A1 and incorporated herein by reference. By subjecting the loaded material comprising said bola SL with an alkaline agent such as NaOH. The pH of the alkaline agent is preferably higher 8, more preferably higher than 12. It has been found that a higher pH improves processing times by increasing the reaction rate. The adsorbent is typically washed with water to remove sophorose and base followed by desorption of acidic sophorolipids.

In still another setup, during the process of the present invention, the glycolipid-containing composition is passed through a reactor that comprises the polymeric adsorbent.

In an embodiment, the polymeric resin as described herein can be mixed with the glycolipid-containing mixture IM in a reactor wherein the process according to the invention is carried out. In a preferred embodiment, the glycolipid-containing composition is passed over a column packed with the polymeric adsorbent during the process according to the different embodiments of the invention. In another embodiment, Lactonic SL are absorbed on the absorbent in the process unit and are modified with an alkaline agent to form acidic SL. The adsorbent is typically washed with water to remove acetate and base.

The process according to the present application can be used for isolation of glycolipids in food industry, cosmetic industry, pharmaceutical industry, environmental protection, or energy-saving industry.

### EXAMPLES

The present invention is described below more specifically with reference to Examples. However, the invention is not limited thereto or thereby, and various modifications are possible within the spirit of the present invention by a person skilled in the art. The current invention is illustrated by way of the following examples.

The concentrations of the liquids in the examples are typically expressed as vol. %. This means the volume of the liquid which may be mixed and/or diluted with other liquids to form a mixture divided by the total volume of the mixture.

### Example 1

### Purification and Isolation of Acidic Sophorolipids (ASL) and Bola Sophorolipids (BSL) with Adsorption Resins from a Complex Mixture

Initial resin screening was performed by mixing resins (polystyrene, styrene-divinylbenzene, polymethacrylate, acrylic ester, dextran based and bituminous coal) with a solution containing SLs. The resins were first sequentially equilibrated with RO water, ethanol and again RO water. Every step, the mixture was placed in an incubator for 15 minutes at 200 rpm. By comparing the concentration of SLs in the liquid before and after the addition of the resin, the loading capacity was estimated. Subsequently, the resins were removed from the mixture by filtration through a 100 μm cut-off sieve and washed with RO water until the conductivity of the wash water reached <100 μS/cm. This ensures that all hydrophilic impurities are removed like sugars and salts.

All the resins were able to retain sophorolipids, also abbreviated herein as SL or SLs, to some extent. The adsorption resins were mixed with acetone for 30 minutes in an incubator at 200 rpm, resulting in recoveries up to 77%. Other solvents such as ethanol, isopropanol and ethyl acetate were also able to elute the SLs.

With a chemically brominated polystyrene-based resin, it was possible to elute all bola SLs without any acidic SLs using 40-50% isopropanol. Although also possible with crosslinked polystyrene resins, the difference in affinity seems smaller, making the separation more difficult.

Bituminous coal only adsorbed acidic SLs and no bola SLs, but the loading capacity was rather low so it would be more suited as a polishing step at the end of the process to remove low amounts of remaining acidic SL impurities in bola SL product.

Although bola SLs are not charged, they are also adsorbed by the hydrophobic backbone of ionic resins. This property can be used to separate acidic and bola SLs, by first eluting the bola SLs with a solvent and subsequently eluting the acidic SLs by regenerating the ion exchange resin.

Ion exchange resins were screened by mixing a SLs solution with each resin. All ionic resins could retain both SLs, it was observed that the weak anionic crosslinked polystyrene resins had an acceptable loading capacity. The bola SLs could be eluted with 70% isopropanol as with crosslinked polystyrene adsorption resins, while the acidic SLs remained associated with the resin by ionic bonding, even when applying 96% isopropanol. The same principle can be applied to remove fatty acids from bola SLs or acidic SLs from lactonic SLs. Acetic acid (CH₃COOH) and citric acid (C₆H₈O₇) could partially remove the acidic SLs. The best results were obtained with a 5% hydrogen chloride (HCl) in water solution.

Additional elution tests were performed using a vacuum manifold, which holds small 10-15 mL columns filled with resin. A selection of adsorption resins was sequentially equilibrated with: RO water, isopropanol and RO water. Next, cell-free supernatant containing SLs produced by fermentation was added to each column. The columns were placed in an incubator for 15 minutes at 30° C. and 200 rpm. This incubation step was repeated for every loading, elution and washing step. The vacuum chamber was used to collect the fractions in 15 mL test tubes for every step. Washing steps were performed with RO water and elution with a 50-96% isopropanol solution. Even with only a short contact time of 15 minutes, loading capacities of up to 10% were registered for chemically brominated polystyrene resins.

The most suited and techno-economic relevant polystyrene resin was tested in glass lab scale columns with a volume of 300 mL. Samples were taken periodically to follow the SLs concentration, conductivity, pH and brix. The protein concentration was determined in every fraction using the standard BCA protein assay. The experiments always ran through the following sequence:
Wash with RO water
Condition resin (new resin only)
Loading of the product
Wash with RO water until conductivity <100 μS/cm
Elute product with 50-100% solvent (e.g. ethanol)
   (Regeneration of the Resin with 90-100% Solvent e.g. Ethanol)

Cell-free fermentation broth containing mainly bola SLs and some non-acetylated acidic SLs was treated with the method described above. Using a polystyrene resin and eluting with 96% ethanol, a loading capacity of more than 7.5% and a recovery of more than 80% was obtained. By extending the elution phase, this recovery could be increased even more. The purity can also be increased, because during elution a significant peak in proteins was detected. Said instances are further described in EXAMPLE 2.

### Example 2

### Purification of Acidic SLs and Bola SLs with Adsorption Resin from Proteins and Colour Pigments

When high ethanol concentrations are used to elute SLs after rinsing the product loaded polystyrene resins with RO water, all SLs desorb but also some proteins as described in EXAMPLE 1. Most of the proteins and colour pigments are already removed during loading and RO water washing steps, but more hydrophobic proteins and colour pigments remained due to their affinity with the resin. The 300 mL experiment in EXAMPLE 1 was repeated, but now the resin was washed with lower ethanol concentrations. More proteins and colour pigments could be removed by first washing the resin with 25-30% ethanol, which is sufficient to eliminate most of the color pigment and proteins but low enough to not desorb any of the SLs. The SLs were then eluted with 50-96% ethanol.

An increased purity of the SLs was obtained by applying the following sequence, without impacting the high recovery:
Wash with RO water
Condition resin (new resin only)
Loading of the product
Wash with 25-30% ethanol/isopropanol
Wash with RO water until conductivity <100 μS/cm
Elute product with 50-100% ethanol/isopropanol
(Regeneration of the resin with 90-100% solvent e.g. ethanol)

The above-mentioned adsorption step can replace or be added to a glycolipid purification process, where an increased purity is desired.

This method was also evaluated on a 30 L pilot scale column containing a polystyrene resin absorbent. The fermentation broth was subjected to microfiltration to remove cells and this filtrate was then treated with the adsorption resin. To elute the SLs, 96% ethanol was used to maximize recovery and fully regenerate the column all in one step. Systematically a loading capacity of more than 8% was registered and a recovery of more than 95% bola SLs was obtained. Comparing this purification strategy to a combination of 50 kDa and 10 kDa PES ultrafiltration purification method on the same fermentative produced cell-free filtrate, both the recovery and purity can be increased significantly as shown in table 1.

TABLE 1

| | Ultrafiltration | Adsorption |
|---|---|---|
| | DSP | DSP |
| SLs recovery (%) | >70% | >95% |
| Proteins (% w/w DM) | 5.6% | 1.1% |
| Glucose (% w/w DM) | 2.3% | 0.3% |
| Oil (% w/w DM) | 0.98% | <0.01% |
| Total purity (% w/w | 91% | 98.6% |
| DM) | | |

Finally, the adsorbent was used to purify SLs from a 15 m³ fermentation. Cells were removed with microfiltration and the filtrate was used to load a 1.1 m³ column with a polystyrene resin. A solution of 70% isopropanol was used to elute the SLs. Several batches were performed, giving an overall recovery of 95% for the bola SLs.

### Example 3

### Purification of Lactonic SL (Non-Water-Soluble) with Adsorption Resins from a Complex Mixture

Samples were prepared containing a mixture of acidic and lactonic SLs with varying degrees of acetylation. Diacetylated lactonic SLs were most prominently present. Nine different adsorption resins (polystyrene, styrene-divinylbenzene, acrylic ester and polymethacrylate based) were screened with both fermentation broth and supernatant, the latter obtained by centrifugation of the same broth.

Laboratory scale tests were performed using a vacuum manifold, which holds small 10-15 mL columns, filled with resin. Before loading, the resins were rinsed with RO water and shaken for 15 minutes at 200 rpm. After removing the water by applying vacuum, the resins were loaded with broth or supernatant and shaken for 30 minutes at 200 rpm. The resins were washed with RO water to remove hydrophilic impurities before elution. A solution of 70% isopropanol in water was used as eluent. A styrene-divinylbenzene based resin showed some adsorption, but the best results were obtained with polymethacrylate and acrylic ester-based resins, reaching loading capacities of up to 8%. Resins with higher surface area were able to adsorb more SLs, but elution of the SLs proved to be significantly harder.

Several other solvents were evaluated on a mixture of acidic SLs and lactonic SLs dissolved in water. The reference elution solvent was 70% isopropanol. Both SLs could be eluted by 70% ethanol, 70% methanol, 100% butanol, 100% acetonitrile, 70% acetone and 100% tetrahydrofuran, with butanol performing better slightly than isopropanol. Apolar solvents, such as hexane and heptane did not elute any SLs and could therefore be suitable to separate free fatty acids, fatty alcohols and oils during the adsorption.

After screening resins for adsorption capacity and elution efficiency, the scale was increased to a 300 mL column. Experiments were performed using the same fermentation broth and supernatant containing SLs. The column was filled with a polymethacrylate based adsorption resin. The column was rinsed with RO water until a conductivity below 20 μS/cm before loading the broth or supernatant. Before elution, the resins were washed with RO water until a conductivity of 50 μS/cm. Elution was carried out with a 70% isopropanol solution.

Due to promising results, lactonic SLs purification was tested on a 5 L glass column. The 5 L column was filled with a polymethacrylate based adsorption resin. The resin was rinsed with RO water until the effluent conductivity dropped below 20 μS/cm.

Fermentation broth was mixed with an equal amount of 96% ethanol to dissolve as much lactonic SLs as possible. The broth was then mixed with diatomite filter aid and poured over a filter bed, to remove all cells and debris. The ethanol in this filtrate was evaporated before loading onto the resin to increase the adsorption efficiency. After loading the column, the resins were washed with RO water until the conductivity dropped below 50 μS/cm. Elution was carried out with a 70% isopropanol solution. Samples were taken every 5 minutes to follow the SLs concentration, conductivity, pH and brix. The overall SL recovery amounted to 89%.

### Example 4

### Purification and Isolation of Bola Sophorosides (BSS), Alkyl Sophorosides and Alcohol Glucosides with Adsorption Resins from a Complex Mixture

Laboratory scale 10-15 mL columns were packed with a polystyrene based resin and rinsed with RO water. Subsequently, cell-free filtrate was obtained with microfiltration of the fermentation broth at elevated temperatures. This filtrate contained alkyl sophorosides (alkyl SSs), alcohol glucosides (alcohol GSs) and bola sophorosides (bola SSs). After loading the filtrate onto the column, the resins were washed with RO water until a conductivity lower than 100 μS/cm. Different solvent mixtures, containing Methyl tert-butyl ether (MTBE) and isopropanol or a mixture of heptane, ethyl acetate, methanol, butanol and water were added to the resin, shaken for 30 minutes at 200 rpm and subsequently eluted.

A mixture of 20% heptane (water solubility of 0.0022 g/100 ml at 25° C.), 27.5% ethyl acetate (EA) (water solubility of 8.7 g/100 ml at 20° C.), 2.5% butanol, 2.5% methanol and 37.5% water seemed to be most selective towards alkyl SSs, eluting 93% while only 10% of alcohol GSs were eluted and no bola SSs. Isopropanol was not selective at all, eluting all three glycolipids.

Methyl tert-butyl ether (MTBE) (water solubility of 4.2 g/100 ml) and diethyl ether (water solubility of 6.9 g/100 ml at 20° C.) showed some selective properties, however with a lower efficiency: 40% alkyl SSs were eluted together with 13% of alcohol GSs and 3% of bola SSs.

The genetically modified Starmerella bombicola strain producing this mixture of sophorosides and glucolipids, also produces de novo fatty acids. These fatty acids elute together with the SSs when using the solvent mixtures mentioned above, so still need to be removed.

After evaporating the solvents, the precipitated SSs were dispersed in RO water and loaded again onto the columns filled with a polystyrene based resin. After washing the columns with RO water, each column was eluted with another solvent (mixture). Following ratios of heptane and ethyl acetate were applied: 100:0, 90:10, 80:20, 60:40 and 40:60. When 100% heptane is used, none of the de novo fatty acid derivatives were removed. The hexane solution appears to be too apolar. A 90:10 solution eluted some of the de novo fatty derivatives and 39.6% of alkyl SSs while the 80:20 solution seemed most promising to remove the majority of the de novo fatty derivatives. Unfortunately, there was also more coelution of 59% alkyl SSs. Both the 60:40 and 40:60 solutions were able to elute all alkyl SSs as well as the de novo fatty derivatives. However, the second ratio also coeluted a fraction of alcohol GSs. More optimization is required, but this experiment shows that separation of de novo fatty acid derivatives, alkyl SSs and alcohol GSs is possible by selective elution from an adsorption resin.

### Example 5

### Purification of Acetylated Bola Sophorosides (ABSS) with Adsorption Resins from a Complex Mixture

An estimation of the adsorption capacity of acetylated bola sophorosides on a polystyrene resin was made by filling 5 mL Eppendorf tubes with resin and loading different known product concentrations (5-12 m/m %) onto the resin. The solutions were prepared by dissolving the required amount of freeze-dried pure product in RO water. After shaking for 45 min at 200 rpm, samples of the solution were taken and analysed with the aid of TLC. If the loading capacity of the resin was exceeded, product spots are found on the TLC plate, indicating not all product was able to bind to the resin.

The conclusion of this experiment was that the adsorption capacity for acetylated bola sophorosides on this resin was between 5 and 6 m/m %. This is significantly lower than the adsorption capacity of the same resin for bola SLs (8 m/m %). The hypothesis was that extra space is occupied by the acetyl groups, allowing less product to adsorb. However, the experiment was repeated and when shaken overnight, even the highest tested bola SSs ratio of 12 m/m % was completely adsorbed to the resin. Possibly multilayer adsorption occurs when sufficient contact time is applied.

Purification of acetylated bola SSs from fermentation broth was first validated on 300 mL columns using the method described in EXAMPLE 1 for bola SLs, and afterwards also on 30 L columns. Prior to use, the columns packed with resin were rinsed with RO water, 96% isopropanol and RO water. After loading cell-free filtrate onto the column, the resins were washed with RO water again until a conductivity of 20 μS/cm. The bola SSs were eluted with 70% isopropanol. A recovery of 88% ABSS was achieved with a purity of more than 95%, which is in line with the results obtained on bola SLs in EXAMPLE 1.

TABLE 2

| Component | Result |
|---|---|
| Acetylated bola SSs recovery (%) | 88% |
| Proteins (% w/w DM) | <1.3% |
| Glucose (% w/w DM) | 0.08% |
| Glycerol (% w/w DM) | <0.1% |
| Free fatty acids (% w/w DM) | <0.33% |
| NaCl (% w/w DM) | <0.5% |
| Total purity (% w/w | >95% |
| DM) | |

### Example 6

### Purification of Sucrose Esters (SE) with Adsorption Resins

Adsorption was evaluated for the E473 sucrose esters that are soluble in warm water. Laboratory scale tests were performed using a vacuum manifold, which holds small 10-15 mL columns, filled with a polymethacrylate based resin. Before loading, the resins were rinsed with RO water and shaken for 15 minutes at 200 rpm. After removing the water by applying vacuum, the resin was loaded with the sucrose ester solution and shaken for 30 minutes at 200 rpm, which was sufficient to adsorb all SEs. The resins were washed with RO water again to remove hydrophilic impurities before elution. Elution with 70% isopropanol was successful, but at a lower recovery than for acidic and lactonic SLs, which can be compensated for by increasing the isopropanol concentration or elution time.

### Example 7

### Chemical (Partial) Hydrolysis of Glycolipids

### (1) on Column (Partial) Hydrolysis of Bola SLs to Produce Acidic SLs and Sophorose

Bola SLs have an ester bond that is sensitive to (alkalic) hydrolysis. The glycosidic bond can withstand alkalic hydrolysis, so the resulting products are non-acetylated acidic SLs and sophorose, which are both interesting molecules. By performing the hydrolysis on column, acidic SLs and sophorose can be produced and purified with the same unit operation, even when starting from a complex mixture such as cell-free fermentation broth containing bola SLs. As the purification happens during the same unit operation, partial hydrolysis can also be performed to obtain a desirable ratio between bola SLs and acidic SLs. Sophorose will elute during the hydrolysis. Sophorose can be further purified by an ion exchange, nanofiltration or dialysis polishing step to remove the ions and salts. A preferred conversion of bola SL into acidic SL and sophorose is illustrated below.

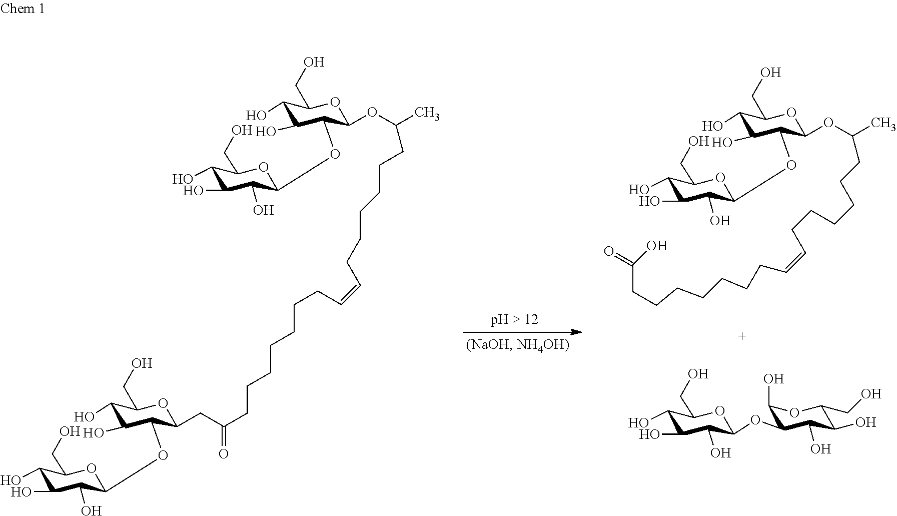

Sodium hydroxide and ammonia hydroxide were evaluated as bases for the chemical hydrolysis by first mixing purified bola SLs with several concentrations (respectively 0.01-0.5 M and 0.1-1 M) of each base and shaking for 1 hour at 200 rpm. When using NaOH, a minimum concentration of 0.05 M or maintaining pH 12 is required to obtain complete conversion. For NH₄OH 1 M is required, significantly higher. The advantage of using NH₄OH would be that it is volatile and can be stripped from sophorose, so no additional complex purification is required. The volatility is also a disadvantage, as it causes irritating ammonia vapors during processing. Hydrolysis already starts at pH 8 but is a lot faster at pH 12. Due to the formation of acidic SLs, the pH will decrease during the hydrolysis and should be increased again to maintain high reaction rates.

TABLE 3

| | Hydrolysis (%) with | Hydrolysis (%) with |
|---|---|---|
| pH | NaOH | NH₄OH |
| 8 | 46.2 | 49.8 |
| 10 | 45.2 | 69.8 |
| 12 | 100 | 82.5 |

Next, hydrolysis was tested on a 300 mL adsorption column, filled with a polystyrene resin. The resin was equilibrated as described in EXAMPLE 1, washed with RO water until a conductivity <50 μS/cm and then a 10 m/m % SL solution per resin was loaded. Two bed volumes at a concentration of 0.1 M and 0.25 M were tested for NaOH and 0.5 M for NH₄OH, all at a flow of around 3 bed volumes per hour. The hydrolysis could be followed due to the rising conductivity and brix-value. This last value is related to the amount of sophorose. The resins were washed with RO water until a conductivity of 50 μS/cm and the SLs were subsequently eluted using 96% ethanol. For both NaOH concentrations no bola SLs were detected, while 0.5 M NH₄OH was not enough to complete the hydrolysis with the current contact time.

The process was scaled up to a 30 L column, using the same SLs load and 2 bed volumes of a 0.1 M NaOH solution at a flow of around 2 bed volumes per hour. During hydrolysis, the pH in the effluent increased to 12.5. After washing with RO water, the SLs were eluted using 50% ethanol. As expected, no bola SLs were detected, indicating complete hydrolysis.

Alternatively, it is also possible to first perform the hydrolysis in a reactor, followed by adsorption, which allows easier follow up and adjustment. However, when using complex compositions such as cell-free fermentation broth, also reactions with other products will occur resulting in unexpected byproducts and higher base consumption. Hydrolysis before adsorption is therefore more likely to be used on more pure and already isolated products.

### (2) on Column Chemical (Partial) Hydrolysis of Lactonic SL

Lactonic SLs are also susceptible to (alkalic) hydrolysis to form acidic SLs. However, fermentative produced lactonic SLs (wild type) are usually double acetylated. The acetate salts and base ions are preferably removed after hydrolysis. Combining hydrolysis and adsorption, non-acetylated acidic SLs can be produced and purified directly from pure product or cell-free fermentation broth containing a mix of acetylated acidic and lactonic SLs.

Hydrolysis tests were performed on lab scale using the same vacuum manifold and methodology described in EXAMPLE 1. The difference is that between the loading and rinsing step, one bed volume 0.1 M NaOH solution was added to the columns. The hydrolyzation reaction was carried out overnight in a thermomixer at 50° C. After 24 hours, the solution containing acetate and salts was removed by applying a vacuum. After rinsing with RO water, the non-acetylated acidic SLs were eluted with 70% isopropanol. Full conversion was observed with these conditions.

### (3) Chemical (Partial) Hydrolysis to Remove Acetylations on Glycolipids

Acetylations can drastically impact the properties of glycolipids. These can be (partially) removed by means of (alkaline) hydrolysis. The acetate salts and base ions are preferably removed after the hydrolysis. By combining hydrolysis and adsorption, glycolipids can be (partially) deacetylated on column and purified directly from pure product or cell-free fermentation broth containing acetylated glycolipids.

### Acetylated Bola SSs

The genetically modified Starmerella bombicola strain used to produce acetylated bola SSs, also makes small amounts of bola SLs. As described in earlier in EXAMPLE 7, on column alkalic hydrolysis can be applied to convert the bola SLs to acidic SLs and sophorose. This will however also remove acetylations, resulting in a different product. The methods in EXAMPLE 1 can then be used to separate the bola SSs from the acidic SLs.

Cell-free broth from a 150 L fermentation containing acetylated bola SLs was hydrolysed to remove acetylations. This was done by adding a 30% NaOH solution until a pH of 12. This solution was incubated for 1 h, continuously monitoring and adjusting the pH to 12, after which the solution was stabilised to pH 4-5 by adding 4.5 M H2SO4.

The hydrolysed solution was loaded onto a 30 L column, containing a polystyrene resin. After rinsing with RO water to a conductivity <50 μS/cm, 70% isopropanol was used to elute the SLs, resulting in pure acetylated bola SSs.

TABLE 4

| Component | Result |
|---|---|
| Proteins (% w/w DM) | <1.3% |
| Glucose (% w/w DM) | 0.04% |
| Glycerol (% w/w DM) | <0.1% |
| Free fatty acids (% w/w DM) | <0.03% |
| NaCl (% w/w DM) | <0.5% |
| Total purity (% w/w | >95% |
| DM) | |

1-32. (canceled)

33. A process for isolating and/or purifying an acetylated or non-acetylated form of a glycolipids from a glycolipid-containing composition containing at least one glycolipid, the process comprising:
providing a process unit with an adsorbent, wherein the adsorbent is a polymeric resin;
contacting the glycolipid-containing composition with the adsorbent to load the polymeric resin with a loaded material, the loaded material comprising an amount of the at least one glycolipid;
treating the adsorbent to recover a predetermined desired type of glycolipid, wherein treating the adsorbent comprises contacting the adsorbent with a preselected recovering liquid to recover the predetermined desired type of glycolipid from the loaded material on the adsorbent; and
acquiring the predetermined desired type of glycolipid recovered from the process unit.

34. The process according to claim 33, wherein treating the adsorbent further comprises contacting the adsorbent with one or more treating liquids to modify the amount of the at least one glycolipid such that a chemical modification and/or an enzymatic modification of the glycolipids adsorbed on the polymeric resin occurs before desorption of the glycolipids from the polymeric resin, the chemical modification and/or enzymatic modification of the adsorbed glycolipids being selected from chemical and/or enzymatic hydrolysis of ester bonds, chemical and/or enzymatic hydrolysis of glycosidic bonds, esterification, etherification, glycosylation, polymerisation, amidation, amination, reductive amination, quaternization, oxidation, or epoxidation.

35. The process according to claim 34, wherein the one or more treating liquids are selected from methanol, ethanol, propanol, isopropanol, butanol, hexane, heptane, ethyl acetate, KOH, NaOH, NH₄OH, water, RO water, or combinations thereof.

36. The process according to claim 33, wherein treating the adsorbent further comprises:
contacting the adsorbent with one or more treating liquids to modify the at least an amount of glycolipids by contacting the adsorbent with an alkaline reagent, to at least partially modify, the glycolipids loaded on the adsorbent.

37. The process according to claim 36, wherein the one or more treating liquids are selected from methanol, ethanol, propanol, isopropanol, butanol, hexane, heptane, ethyl acetate, KOH, NaOH, NH₄OH, water, RO water, or combinations thereof.

38. The process according to claim 33, wherein the preselected recovering liquid is selected from ionic liquids, liquid carbon dioxide, supercritical solvents, ethyl acetate, methanol, isopropanol, acetone, ethanol, heptane, tert-butyl-methyl ether, diethylether, acetonitrile, phenoxyethanol, benzyl alcohol, phenethyl alcohol, hydrocinnamyl alcohol, tetrahydrofurfuryl alcohol, dimethylisosorbide, methyl salicylate eugenol, linalool, hexanol, glacial acetic acid, dimethylcarbonate, dipropyleneglycol methyl ether, ethyl-lactate, butyl-lactate, amyl-lactate, ethylhexyl-lactate, or combinations thereof.

39. The process according to claim 33, wherein treating the adsorbent comprises:
contacting the adsorbent with a treating solution having a first concentration of a treating liquid;
contacting the adsorbent with a recovering solution having a second concentration of a recovery liquid;
wherein:
the first concentration is such that that essentially all of the glycolipids within the loaded material are not removed from the adsorbent;
the second concentration is such that essentially all of the glycolipids within the loaded material from the adsorbent are removed from the adsorbent.

40. The process according to claim 33, wherein treating the adsorbent further comprises:
contacting the adsorbent with a first treating liquid, to at least partially modify the glycolipids loaded on the adsorbent.

41. The process according to claim 33, wherein:
the glycolipid comprises acetylated and/or non-acetylated bola sophorolipids; and
the process converts the acetylated and/or non-acetylated bola sophorolipids into acetylated and/or non-acetylated acidic sophorolipids and acetylated and/or non-acetylated sophorose.

42. The process according to claim 33, wherein:
treating the adsorbent comprises contacting the adsorbent with a second treating liquid that is an apolar solvent, to remove a second fraction of one or more secondary components from the loaded material;
the second fraction is selected from free fatty acids, fatty alcohols, triacyl glycerides, dicarboxylic acids, diols, alkanes, and/or oils; and
the second treating liquid is chosen from hexane, heptane, or methyl tert-butyl ether.

43. The process according to claim 33, wherein:
treating the adsorbent comprises contacting the adsorbent with a third treating liquid to remove a third fraction of one or more secondary components from the loaded material; and
the third fraction a hydrophilic impurity selected from sugars, proteins, peptides, polyols, organic acids, inorganic acids, carbohydrates, or salts.

44. The process according to claim 33, wherein the polymeric resin is selected from polymethacrylate resin, acrylic resin, polystyrene resin, or combinations thereof.

45. The process according to claim 33, wherein the polymeric resin is selected from styrene-divinylbenzene, polymethacrylate, chemically brominated polystyrene, acrylic ester, polystyrene, crosslinked polystyrene, or methacrylic porous polystyrene-divinylbenzene.

46. The process according to claim 33, wherein the polymeric resin is chemically brominated polystyrene.

47. The process according to claim 33, further comprising:
recycling at least one of the recovering liquid or the treating liquid; and
optionally recycling the washing liquid.

48. The process according to claim 33, wherein the glycolipid-containing composition is an end-product of a plant extract or a fermentative, enzymatic, or chemical production method.

49. The process according to claim 33, wherein the process is performed in combination with a fermentation process in an in-situ product recovery set-up.

50. The process according to claim 33, wherein the glycolipids are selected from acetylated or non-acetylated forms of bola sophorolipids, bola sophorosides, alkyl sophorosides, alcohol glucosides, sucrose esters, bola glucosides, alkyl glucosides, glucolipids, or combinations thereof.

51. The process according to claim 33, further comprising passing the glycolipid-containing composition through a reactor that comprises the adsorbent.

52. The process according to claim 33, further comprising passing the glycolipid-containing composition over a column packed with the adsorbent.
